(19) United States
(12) Patent Application Publication
(10) Pub. No.: US 2008/0171929 A1
(43) Pub. Date: Jul. 17, 2008

(54) METHOD FOR STANDARDIZING SPACING BETWEEN ELECTRODES, AND MEDICAL TAPE ELECTRODES

(76) Inventor: Jefferson J. Katims, Towson, MD (US)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 11/971,545

(22) Filed: Jan. 9, 2008

Related U.S. Application Data
(60) Provisional application No. 60/884,471, filed on Jan. 11, 2007.

(51) Int. Cl. A61B 5/04

(52) U.S. Cl. 600/391

(57) ABSTRACT

Standardization between paired electrodes is maintained in a medical device without needing a Mylar spreader, such as by forming the paired electrodes integrally with a tape part.

### RELATED APPLICATION

This application claims benefit of U.S. provisional application Ser. No. 60/884,471 filed Jan. 11, 2007 titled “Tapetrode Electrodes” by Jefferson Katims.

### FIELD OF THE INVENTION

This invention relates to medical electrode devices, especially medical tape electrodes and medical devices including paired electrodes in which a certain inter-electrode distance is wanted to be maintained.

### BACKGROUND OF THE INVENTION

Medical electrodes have been used in a variety of configurations, for various uses. A preferred application uses a pair of medical electrodes, of which a preferred example of medical electrodes to pair is the Goldtrode® (registered trademark of Neurotron, Inc.) electrode. The electrodes are snapped-in to wiring. In a typical application method, to a finger, medical tape (such as Softape®) is applied to one electrode holder, and the first electrode is applied to one side of a finger; the second electrode is applied at the other side of the finger and the medical tape is wrapped around the finger to keep the electrodes on the finger. The medical tape might partly encircle the finger, or alternately wrap around the electrode placement and encircle the finger. Another site for positioning paired Goldtrode® electrodes is a trigeminal nerve test site, by using medical tape to tape the paired electrodes on the face. A further site for positioning paired Goldtrode® electrodes is a lumbar test site on the outside of a big toe.

In these various positions, the paired Goldtrode® electrodes are being held a distance apart using another separate piece, a Mylar spreader, inserted to form a combination before the taping-on using the medical tape is accomplished.

Referring to the literature, the following are mentioned:

U.S. Pat. No. 4,008,721 issued Feb. 22, 1977 to Burton (Medtronic, Inc.), for “Tape electrode for transmitting electrical signals through the skin.”

U.S. Pat. No. 4,067,342 issued Jan. 10, 1978 to Burton (Medtronic, Inc.), for “Tape electrode.”

U.S. Pat. No. 4,141,366 issued Feb. 27, 1979 to Cross, Jr. (Medtronic, Inc.), for “Lead connector for tape electrode.”

U.S. Pat. No. 4,243,052 issued Jan. 6, 1981 to Bailey (Stimtech, Inc.), for “Disposable electrode.”

U.S. Pat. No. 4,243,051 issued Jan. 6, 1981 to Witteman (Johnson & Johnson), for “Disposable electrode.”

U.S. Pat. No. 4,305,402 issued Dec. 15, 1981 to J. Katims, for “Method for transcutaneous electrical stimulation.”

U.S. Pat. No. 4,503,863 issued Mar. 12, 1985 to J. Katims, for “Method and apparatus for transcutaneous electrical stimulation.”

J. Katims, D. M. Long, L. K. Y. Ng, “Transcutaneous Nerve Stimulation: Frequency and Waveform Specificity in Humans,” Appl. Neurophysiol. 49: 86-91 (1986).

U.S. Pat. No. 4,580,339 issued Apr. 8, 1986 to Ioffe (Empi, Inc.), for “Method for fabricating a disposable electrode for transcutaneous nerve stimulator.”

U.S. Pat. No. 4,640,289 issued Feb. 3, 1987 to Craighead (3M), for “Biomedical electrode.”

Katims, J. J., Rouvelas, P., Sadler, B., Weseley, S. A. Current Perception Threshold: Reproducibility and Comparison with Nerve Conduction in Evaluation of Carpal Tunnel Syndrome. Transactions of the American Society of Artificial Internal Organs, Volume 35:280-284, 1989.

J. Katims, D. Taylor and S. Weseley, “Sensory Perception in Uremic Patients,” ASAIO Transactions, 1991, 37:M370-M372.

Katims, J. J., Patil, A., Rendell, M., Rouvelas, P., Sadler, B., Weseley, S. A., Bleecker, M. L. Current Perception Threshold Screening for Carpal Tunnel Syndrome. Archives of Environmental Health, Volume 46(4):207-212, 1991.

U.S. Pat. No. 5,143,081 issued Sep. 1, 1992 to Young et al., for “Randomized double pulse stimulus and paired event analysis.”

U.S. Pat. No. 5,806,522 issued Sep. 15, 1998 to Katims, for “Digital Automated Current Perception Threshold (CPT) determination device and method.”

U.S. Pat. No. 5,985,990 issued Nov. 16, 1999 to Kantner et al. (3M Innovative Properties Co.), for “Use of pendant free-radically polymerizable moieties with polar polymers to prepare hydrophilic pressure sensitive adhesive compositions.”

U.S. Pat. No. 6,016,451 issued Jan. 18, 2000 to Sanchez-Rodarte for “Neurological stabilizer device.”

U.S. Pat. No. 6,121,508 issued Sep. 19, 2000 to Bischof et al. (3M Innovative Properties Co.), for “Polar, lipophilic pressure-sensitive adhesive compositions and medical devices using same.”

U.S. Pat. Application No. US 2002/0055688 published May 9, 2002 by J. Katims, titled “Nervous tissue stimulation device and method.”

U.S. Pat. Application No. 2005/192567 published Sep. 1, 2005 by J. Katims, is titled “Nervous tissue stimulation device and method.”

U.S. Pat. No. 7,062,319 issued Jun. 13, 2006 to Ihme et al. (Innokas Medical Oy), titled “Method and arrangement for determining suitable treatment frequency and/or intensity.”

U.S. Pat. No. 7,206,632 issued Apr. 17, 2007 to King (Medtronic, Inc.), for “Patient sensory response evaluation for neuromodulation efficacy rating.”

### SUMMARY OF THE INVENTION

The present inventor has identified the need to apply medical tape and arrange the medical tape, electrode and Mylar spreader components successfully using the medical tape as the biggest impediment to more widespread acceptance of paired medical electrodes. The present inventor has removed the need for a user applying the paired medical electrodes to: separately obtain medical tape, maneuver a spreader (such a Mylar spreader or other rigid or semi-rigid spread) between the paired electrodes, and maneuver and position the medical tape, the spreader and the paired electrodes with respect to the site on the body.

In one preferred embodiment, the invention provides a spreader-free method of standardizing a distance between a first electrode and a second electrode, comprising: forming a tape part (preferably a tape part that is non-conductive) integrally with the first electrode and the second electrode, whereby the distance between the first electrode and the second electrode is standardized without disposing a rigid spreader between the first electrode and the second electrodes, such as, e.g.: a spreader-free standardizing method comprising standardizing the distance between the first electrode and the second electrode to a certain distance (such as, e.g., 17 mm); a spreader-free standardizing method further comprising applying the paired electrodes having the standardized distance therebetween to an ear, a digit, a body surface of a person or an animal or a position in a set-up of equipment; etc.

In another preferred embodiment, the invention provides an electrode structure, consisting essentially of: a tape electrode including a tape part (preferably a tape part that is non-conductive) integrally with a pair of electrodes; wherein no rigid spreader is included in the medical electrode; such as, e.g.: an electrode structure wherein the tape electrode is configured to receive a digit or an ear, or is configured to be applied to an external body surface, or is otherwise configured for use medically, or is configured to be operated in carrying out research, or is configured to receive a body part of an animal; an electrode structure wherein the paired electrodes are non-sticky independent of the tape part; an electrode structure in which other than the tape part no further adhesive-containing component or sticky component is included.

The invention in a further preferred embodiment provides an electrode structure, consisting essentially of: a tape electrode including a tape part (preferably a tape part that is non-conductive) integrally with a pair of electrodes; wherein a fixed distance between the paired electrodes is maintained; such as, e.g.: an electrode structure wherein the fixed distance is 17 mm and the paired electrodes are medical electrodes.

In another preferred embodiment the invention provides a spreader-free method of positioning paired medical electrodes, consisting essentially of: applying a single piece to a body surface or body part, wherein the single piece includes the paired medical electrodes integral with a tape part (preferably a tape part that is non-conductive).

The invention in a further preferred embodiment provides a method of positioning a pair of medical electrodes, consisting essentially of: applying the pair of medical electrodes to a body surface or body part in about 1 second or less, while achieving a desired standardized distance between the paired medical electrodes; such as, e.g.: a positioning method wherein the pair of medical electrodes are part of a single piece construction requiring no pre-assembly before the step of applying to the body surface or body part.

In another preferred embodiment the invention provides a method of stimulating a body area too small to practically have positioned thereon two electrodes, comprising: applying onto the body area a single electrode, the single electrode being formed on a tape (preferably a tape that is non-conductive); and applying onto the body but outside the body area a dispersion electrode.

### BRIEF DESCRIPTION OF THE DRAWINGS

The invention may be appreciated further with reference to the figures, without the invention being limited to the figures. The figures are not drawn to scale.

FIG. 1 is a top view of an exemplary embodiment of an inventive tape electrode 10 comprising electrodes 1 and 1′ and tape 2, with inventive tape electrode 10 on backing 3. “A” refers to the horizontal top of tape electrode 10 in FIG. 1; “B” refers to the horizontal bottom of tape electrode 10 in FIG. 1.

FIG. 1A is a bottom view of tape electrode 10 from FIG. 1 after tape electrode 10 has been unpeeled from backing 3 and turned over. FIG. 1A is the underside of tape electrode 10 in FIG. 1.

FIG. 1B is a cross-sectional view corresponding to FIG. 1 viewed along “B” when the tape electrode 10 has been unpeeled from the backing 3 (such as a paper backing). The thickness t of the tape electrode 10 may be, for example, about 2 mm. The electrodes 1, 1′ are at least slightly thicker than the tape 2 in order to protrude beyond the tape 2 and each be snappable into an electrode cable (not shown) such as an electrode cable useable with a conventional Goldtrode® electrode. Electrode 1 is snappable into an electrode cable at surface 1S; electrode 1′ is snappable into an electrode cable at surface 1S′. The shapes of the electrodes 1, 1′ in FIGS. 1, 1A, 1B are representational and the electrodes are not limited to such shapes. The electrode cable C is built into the snap connector.

FIG. 2 shows the tape electrode 10 of FIGS. 1, 1A, 1B after the electrode cables C have been snapped-in after which the tape electrode 10 has applied onto a finger F. Each electrode 1, 1′ is hidden by a respective cable C; electrodes 1, 1′ (not visible in FIG. 2) are on opposite sides of the finger F.

FIG. 3 shows a single-electrode tape electrode 10′ applied to a person's face with a dispersion electrode DE applied to the person's palm.

### DETAILED DESCRIPTION OF THE PREFERRED EMBODIMENT OF THE INVENTION

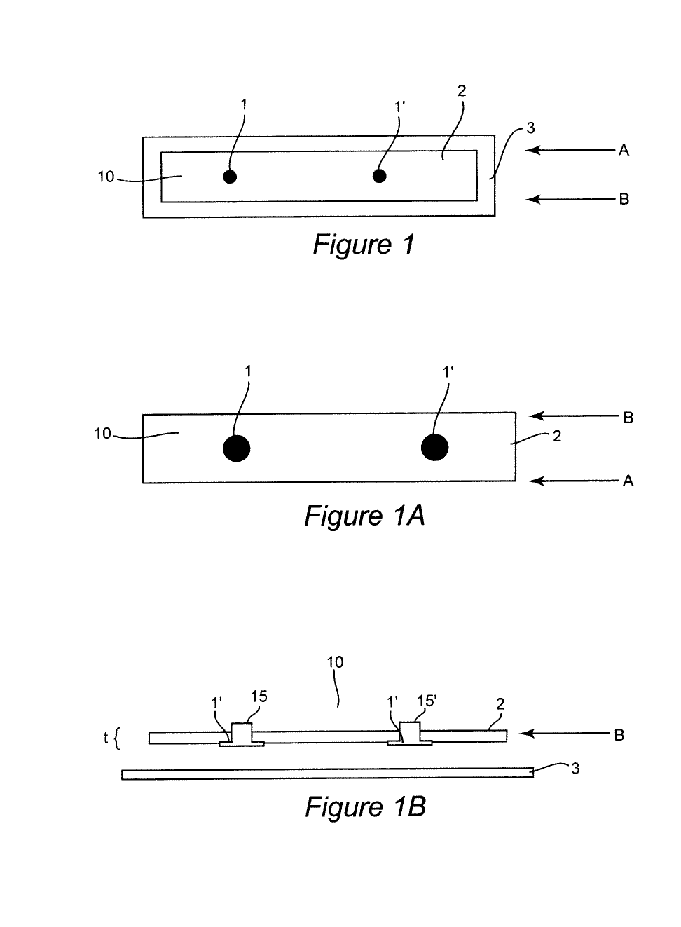

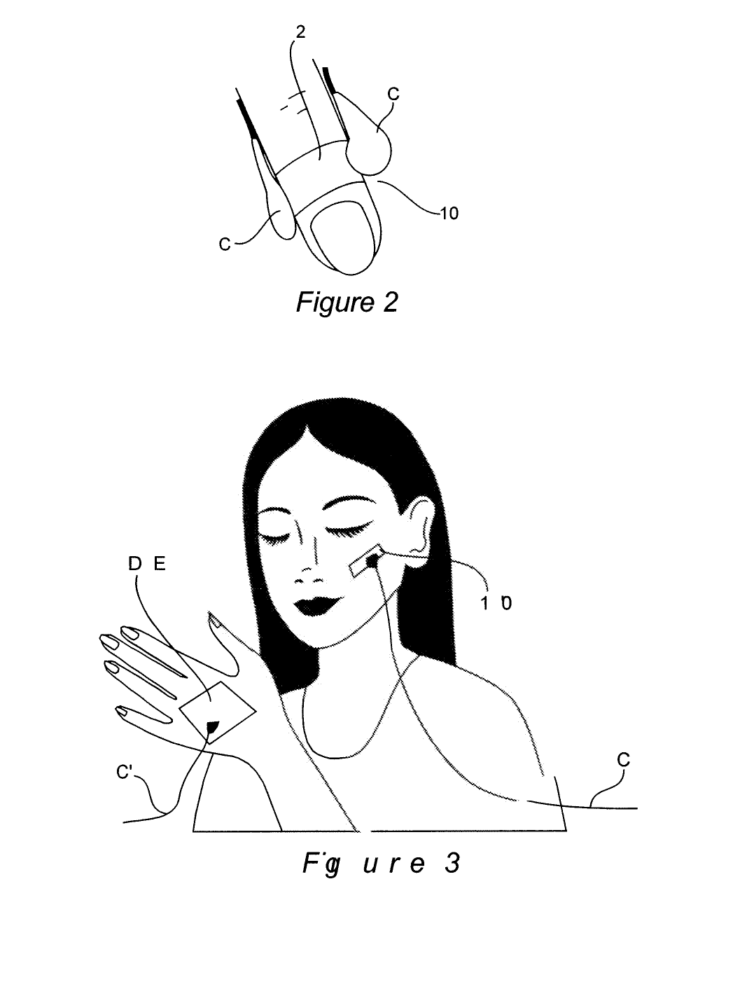

Referring to FIGS. 1, 1A, 1B and 2, a standardized distance between electrode 1 and electrode 1′ is maintained by providing electrodes 1, 1′ integral with tape 2 during a manufacturing operation.

Preferred examples of electrodes used in the invention are, e.g., gold-plated electrodes, snap electrodes, and other medical-use electrodes, etc. Preferably an electrode used in the invention is a snap electrode that snaps into an electrode cable, providing the advantage that the inventive electrode structure then need not include any permanently attached wire. Preferably, an inventive electrode structure excludes any permanently attached wire.

As preferred examples of tape to use in the invention are mentioned, e.g., medical tape, non-conductive tape, and other non-conductive tapes applicable to the human body, etc.

For constructing an electrode (such as electrodes 1, 1′ in FIG. 1) integral with tape (such as tape 2 in FIG. 1), the following steps may be performed: calibrated hole-punching (such as using a hole-punch or die-stamp) of commercially available medical tape, followed by inserting an electrode (such as a Goldtrode® electrode) into a hole so that the narrow part of the electrode is at the non-sticky side of the tape. In one embodiment, the electrode-insertion into holes may be performed before the tape is given to the user who will apply the tape electrode. Alternately, in another embodiment, the user who will apply the tape electrode may be supplied with hole-punched tape with separate electrodes that have not yet been inserted into the tape, for the user to insert into the holes immediately before use.

The inventive paired electrode device 10 (FIG. 1) during manufacture preferably has backing 3 mounted onto tape 2, tape 2 being easily peeled from the backing 3 immediately before end-use application, such as application onto a finger F (FIG. 2) or any skin test site.

A paired electrode medical device such as tape electrode 10 may be used, e.g., by application to a body part such as, e.g., a finger F (FIG. 2) or any skin test site. Preferably a water-based hypo-allergenic chloride-free electrolyte-containing gel is applied to the electrode before the electrode is brought in contact with the skin surface. The gel serves as an electrical-conducting medium between the electrode and the skin.

The invention may be further appreciated by considering the following examples, the invention not being limited to the following examples.

### Comparative Example 1

### U.S. Pat. No. 5,806,522

Paired medical electrodes useable at various body sites are disclosed in U.S. Pat. No. 5,806,522 to Katims.

### Comparative Example 1A

### Goldtrode® Electrodes with Spreader

The electrode is composed of a brass base material, plated with nickel and 22 K gold. The spreader is clear Mylar polyester. The electrodes are 1 cm in diameter snap fasteners. The stimulating surface is 1 cm in diameter. The snap fastening component is a cylinder approximately 3 mm in diameter and 3 mm in length. The electrodes are attached to the clear mylar spreader and then the electrodes are snapped into the electrode cable. The weight of the two Goldtrode® electrodes including the Mylar spreader is approximately 3 grams.

Such stimulating electrodes, known as Goldtrodes®, were gold-plated snap fasteners connected via a mylar spreader, and then snapped into the electrode holders. A strip of sticky tape was connected to the back of one of the electrode holders, and the electrode was then placed on the side of the finger. The tape was then wrapped around the finger and the Goldtrode® snap electrodes' mylar spreader was connected to the electrode cable, which was then placed on the other side of the finger, and the electrode tape was brought around to cover the second electrode.

### Inventive Example 1

### Tapetrode

A tape electrode device according to FIGS. 1, 1A, 1B is constructed. The inventive Tapetrode requires less parts compared to Comparative Examples 1, 1A in which a mylar spreader is needed. Also, the inventive Tapetrode is easier to use or apply or hold in place.

The tape 2 and the electrodes 1, 1′ are integral combined components in the present invention. It is easier to use of the electrodes 1, 1′ and tape 2 as one as in the present invention in contrast to the conventional devices in which electrodes and tape were separate components. Previously the application of the separate tape used with the electrodes was the biggest impediment to usage of the paired electrode technology of Comparative Examples 1, 1A and now this problem has been solved. The Tapetrode of Inventive Example 1 may be applied as easily as a band-aid, while the conventional device was not so easily applied.

### Inventive Example 2

### Single Electrode Integral with Tape

A single-electrode tape electrode such as single-electrode tape electrode 10′ (FIG. 3) is manufactured in much the way as electrode 1 of FIG. 1 is formed on tape 2 to produce paired-electrode tape electrode 10 (FIG. 1) but without electrode 1′ (FIG. 1). The electrode used in a single-electrode tape electrode such as tape electrode 10′ (FIG. 3) is snappable to electrode cable C as electrodes 1, 1′ were snappable to electrode cable C when used in a paired-electrode tape electrode.

A single-electrode tape device (such as single-electrode tape device 10′ (FIG. 3) may be used in combination with a dispersion electrode DE by applying the single-electrode tape device to one part of the body (such as the face as in FIG. 3) and applying a dispersion electrode DE elsewhere to another part of the body (such as the palm as in FIG. 3, shoulder, etc.). Such a set-up is advantageous for trying to stimulate tiny areas where two electrodes are impractical. The palm is preferred as a place for positioning the dispersion electrode DE because there is no hair on the palm. An example of dimensions of a dispersion electrode DE is, e.g., 10 cm by 10 cm. A dispersion electrode DE may be about 100 times the surface area of the tape electrode with which it is used, with a relatively greater surface area of the dispersion electrode being preferred in order to dissipate the electrical current.

### Inventive Example 2A

### Diagnostic Tool Regarding Repair of Facial Nerve Damage

The single-electrode tape device of Inventive Example 2 was used for evaluation of function of facial nerve subsequent to repair after trauma. A smaller surface area of the face was covered by using a single gold electrode on the face in combination with a dispersion electrode elsewhere, as compared to the surface area of the face needed to be covered if instead using two gold electrodes on the face. In this example, the inventor pre-punched medical tape and provided the pre-punched tape to the end users with a supply of Goldtrode® electrodes to be inserted into the holes.

While the invention has been described in terms of its preferred embodiments, those skilled in the art will recognize that the invention can be practiced with modification within the spirit and scope of the appended claims.

What I claim as my invention is:

1. A spreader-free method of standardizing a distance between a first electrode and a second electrode, comprising:
forming a tape part integrally with the first electrode and the second electrode, whereby the distance between the first electrode and the second electrode is standardized without disposing a rigid spreader between the first electrode and the second electrodes.

2. The spreader-free standardizing method of claim 1, comprising standardizing the distance between the first electrode and the second electrode to 17 mm.

3. The spreader-free standardizing method of claim 1, wherein the tape part is non-conductive.

4. The spreader-free standardizing method of claim 1, further comprising applying the paired electrodes having the standardized distance therebetween to an ear, a digit, a body surface of a person or an animal or a position in a set-up of equipment.

5. An electrode structure, consisting essentially of:
a tape electrode including a tape part integrally with a pair of electrodes; wherein no rigid spreader is included in the medical electrode.

6. The electrode structure of claim 5, wherein the tape electrode is configured to receive a digit or an ear, or is configured to be applied to an external body surface, or is otherwise configured for use medically, or is configured to be operated in carrying out research, or is configured to receive a body part of an animal.

7. The electrode structure of claim 5, wherein the tape part is non-conductive.

8. The electrode structure of claim 5, wherein the paired electrodes are non-sticky independent of the tape part.

9. The electrode structure of claim 5, provided that other than the tape part no further adhesive-containing component or sticky component is included.

10. An electrode structure, consisting essentially of:
a tape electrode including a tape part integrally with a pair of electrodes; wherein a fixed distance between the paired electrodes is maintained.

11. The electrode structure of claim 10, wherein the fixed distance is 17 mm and the paired electrodes are medical electrodes.

12. A spreader-free method of positioning paired medical electrodes, consisting essentially of:
applying a single piece to a body surface or body part, wherein the single piece includes the paired medical electrodes integral with a tape part.

13. A method of positioning a pair of medical electrodes, consisting essentially of:
applying the pair of medical electrodes to a body surface or body part in about 1 second or less, while achieving a desired standardized distance between the paired medical electrodes.

14. The positioning method of claim 13, wherein the pair of medical electrodes are part of a single piece construction requiring no pre-assembly before the step of applying to the body surface or body part.

15. A method of stimulating a body area too small to practically have positioned thereon two electrodes, comprising:
applying onto the body area a single electrode, the single electrode being formed on a tape; and
applying onto the body but outside the body area a dispersion electrode.
